(19) United States
(12) Patent Application Publication
(10) Pub. No.: US 2004/0143170 A1
(43) Pub. Date: Jul. 22, 2004

(54) Intelligent deception verification system

(76) Inventor: Donald R. DuRousseau, Purcellville, VA (US)

Correspondence Address: Pepper Hamilton LLP, Firm 21269, One Mellon Center, 50th Floor, 500 Grant Street, Pittsburgh, PA, 15219, US

(21) Appl. No.: 10/736,490

(22) Filed: Dec. 15, 2003

Related U.S. Application Data
(60) Provisional application No. 60/435,511, filed on Dec. 20, 2002.

(51) Int. Cl. A61B 5/00

(52) U.S. Cl. 600/300; 600/544; 600/546; 600/509; 600/595; 600/547; 600/529; 600/549; 600/558; 128/920

(57) ABSTRACT

A psychophysiological signal processing system uses mental and physical activity to determine the intent of an examinee to conceal information or deceive an examiner or trained observer. Brainwaves; eye, heart, muscle, and/or speech activity; skin conductance, resistance, and/or impedance; body temperature, position, posture, expression, and/or gestured motion; blood flow and volume; and stress-indicating measures like respiration, blood pressure, heart rate, and/or other such phenomena that can be sensed from the body may be utilized. A computer-adaptive system analyzing one or more of these psychometric data may be used in combination with a virtual reality system presenting stimuli to the examinee to enhance existing polygraph methods used for individual screening, debriefing, identification and/or certification of information, interrogation, and/or the detection of deception. The virtual reality system may present stimuli designed to evoke a particular measurable response from, confound attempts to avoid detection of deception by, or otherwise distract the examinee.

### RELATED APPLICATIONS

This application claims priority to the co-pending U.S. provisional patent application No. 60/435,511, filed Dec. 20, 2002, entitled “Intelligent Deception Verification System,” which is incorporated herein by reference in its entirety.

This application is related to U.S. patent application Ser. No. 10/028,902, filed Dec. 18, 2001, by Donald R. DuRousseau, titled “Method and System for Initiating Activity Based on Sensed Electrophysiological Data,” which is incorporated herein by reference in its entirety.

### FIELD OF THE INVENTION

The present invention generally relates to automated deception detection devices. More specifically, the present invention is directed to a method and system for sensing and processing mental and physical signals from the human body through the use of actively attached, passively contacted, and/or nearby or distant non-contacted sensors that collect information related to the physiological and behavioral activities of an individual or group of individuals for the purpose of determining deceptive intent. Additionally, a preferred embodiment of the present invention relates to the presentation of an immersive multimedia virtual-reality environment to an examinee while his or her behavioral and/or physiological activities are monitored.

### BACKGROUND OF THE INVENTION

The psychophysiological detection of deception (PDD) is a procedure routinely used by the U.S. Department of Defense (DoD), various law enforcement agencies, officers of the court, and others to determine an individual's truthfulness concerning topics of interest. In theory, the examinee's physiologic reactivity varies with personal relevance of presented stimuli and, more so, with attempts to conceal that relevance from the examiner. In the field of PDD, the variability of psychophysiological responses can be detected by measurements of blood pressure, galvanic skin response, heart rate, respiratory rate and volume, electroencephalography (EEG) and evoked potentials, as well as eye activity. Typically, these measures are assessed (visually) by a trained examiner and are subject to considerable subjectivity and variability in accuracy and sensitivity. Increased reactivity, defined as a change in response level to some stimuli but not others, is assumed to reflect the personal relevance of stimuli presented to the examinee.

The typical PDD examination is designed to elicit outwardly observable physiologic responses from the examinee to specific questions regarding topics of interest. These physiologic responses are then subsequently scored by one or more methods and interpreted by the examiner as indicating the truthfulness of the examinee's verbal responses to the questions of interest.

Existing PDD methods require rather large and cumbersome analog polygraph devices. Even those examiners using somewhat portable digital devices must still use separate and bulky sensing, computing, monitoring, and analysis devices.

In addition to the equipment size problem, the science of PDD continues to rely on the interrogation skills of the examiner and on the examiner's subjective visual interpretation of the polygraph data. Unfortunately, there is considerable variability in the accuracy of results across examiners, and human examiners cannot operate as quickly or routinely as automated detection methods. Further, individuals who are trained to use countermeasures such as tongue biting, toe curling, sphincter tightening or mental manipulation of numbers can often defeat examiners. Some researchers have tested the use of physical and mental countermeasures during a control question test technique and found that the countermeasure methods were equally effective at defeating the polygraph test as administered by human examiners. In one study, fifty percent of examinees defeated examiners, and countermeasures were reported as very difficult to detect.

To be more useful in the future, PDD methods must remove the subjectivity of the human examiner by providing automated detection algorithms that can accurately determine when an examinee is attempting to deceive the examiner or subvert the interrogation by using countermeasures. Although commercial automated software systems for analyzing PDD data and rendering decisions have been developed, studies have found that prior methods of computer aided detection are correct only 88 to 91% of the time.

Thus, a need exists for intelligent automated routines that will look for signs of countermeasure use and improve the accuracy of deception detection, preferably to 95% or more.

In addition, a need exists for a method and system used to disrupt the use of countermeasures by an examinee in order to increase the accuracy of deception detection.

Accordingly, it is desirable to provide an improved deception detection device and system.

### SUMMARY OF PREFERRED EMBODIMENTS OF THE INVENTION

In a preferred embodiment, the present invention provides a portable intelligent deception verification system (IDVS) that utilizes (preferably ultra-lightweight) sensor and processing hardware systems and sophisticated signal processing software (or firmware) to acquire and measure psychometric data under real-world conditions.

A preferred embodiment of the present invention also provides an immersive multimodal virtual-reality stimulus presentation system that can be synchronized with the acquisition and measurement of psychometric data.

A preferred embodiment of the present invention also integrates a multichannel signal processing system to record and analyze psychophysiological and physical processes, related to measures of cognition and stress, as well as other processes that are related to blood flow, movement, gestures, expressions, gazes and other such activities.

A preferred embodiment of the present invention also provides specially configured sensor and/or transducer kits packaged to acquire application specific signal sets depending on the accessibility of the examinee. For instance, sensors attached on or near the body may be used when the examinee is present. Cameras, lasers, infrared, and ultra-sound devices, as well as magnetic and radar imagers and other devices, may optionally be used from a distance and not in contact with the body.

A preferred embodiment of the present invention also provides a universal interface to the signal processing system that is modular and allows attachment to many different sensors, transducers, or other such measurement devices or systems.

A preferred embodiment of the present invention also provides a simple mechanism for investigators to include text, speech, sounds, photographs, video details, testimony, and/or other such evidence for use within the immersive multimedia stimulus presentation component of the present invention.

A preferred embodiment of the present invention also uses immersive virtual-reality presentation and analytical signal processing methods that measure and quantify a host of psychometric data and output specific indices that reflect the use of mental and/or physical countermeasures intended to purposely defeat the detection of deception.

A preferred embodiment of the present invention also provides a library of cognitive and stress related signal processing algorithms and methods, which measure and quantify numerous psychometric indices derived from the examinee's mental, physical, physiological, postural, and/or position related activities.

A preferred embodiment of the present invention also collects, processes and communicates psychometric data over a communications system such as the Internet, preferably anywhere in the world, to make it available for review or augmentation at a location remote from the operator or examinee.

A preferred embodiment of the present invention also provides a computer-aided interrogation development system that can be bundled as a Software Developers Kit (SDK) that provides a graphical user interface (GUI) for programming user specific interrogation protocols. The SDK of the present invention can preferably operate within standard operating systems like Microsoft Windows®, UNIX® and LINUX®.

A preferred embodiment of the present invention also includes, with the SDK, subroutines that allow developers to create software with the ability to instantly modify the presentation of multimedia stimuli, based on the psychometric activity measured by the examinee.

A preferred embodiment of the present invention also provides a single sensor, or group of sensors, that may be used to acquire signals from the brain, eyes, skin, heart and/or muscles by providing a means to position sensors in the appropriate regions of the scalp, face, chest and/or body.

A preferred embodiment of the present invention also provides a single lead wire, or a group of lead wires, that may be used to connect to and communicate signals from body-mounted and distant transducer devices used to measure respiration, blood flow, temperature, heart rate, impedance, motion, acceleration, load, pressure and/or other attributes by providing a means to position them in the appropriate regions of the limbs, chest, waist, hips and/or other part(s) of the body.

A preferred embodiment of the present invention provides direct or wireless access to sensors, transducers, and/or other measurement devices that use video, audio, infrared, laser, radar, ultra-sound, radio frequency, microwave, vibration, motion, and/or acceleration to detect deception.

It is to be understood that the invention is not limited in its application to the details of construction and to the arrangements of the components set forth in the description contained herein or illustrated in the drawings. The invention is capable of other embodiments and of being practiced and carried out in various ways. Hence, it is to be understood that the phraseology and terminology employed herein, as well as in the abstract, are for the purpose of description and should not be regarded as limiting.

As such, those skilled in the art will appreciate that the conception upon which this disclosure is based may readily be utilized as a basis for designing other structures, methods, and systems for carrying out the several purposes of the present invention.

### BRIEF DESCRIPTION OF THE DRAWINGS

Aspects, features, benefits and advantages of the embodiments of the present invention will be apparent with regard to the following description and accompanying drawings.

FIG. 1 illustrates several hardware elements of a preferred system embodiment of the invention.

FIG. 2 illustrates an exemplary agent flow control diagram according to an embodiment of the present invention.

FIG. 3 is a block diagram illustrating exemplary elements of a digital processor, memory and other electronic hardware according to an embodiment of the present invention.

### ADDITIONAL DESCRIPTION OF PREFERRED EMBODIMENTS OF THE INVENTION

The present invention uses human-computer interoperability methods in which the analysis of multimodal psychophysiological measures is related to cognition and stress. These cognitive and stress assessment methods are derived using highly constrained spatio-temporal EEG analysis, expert-based heart, eye, muscle, voice, electrodermal, thermal, circulatory, and/or respiratory data processing algorithms, and adaptive neural network (ANN) pattern recognition and classification techniques to identify psychophysiological indices of deceitful or deceptive activity of individuals or groups.

A preferred embodiment of the invention uses wearable computing systems to detect and record brainwave, eye, heart and/or muscle activity; skin conductance, resistance, and/or impedance; body position, posture, expression, and/or gestured motion; speech; and/or body temperature. The systems may also include blood flow sensors, as well as stress measurement sensors that process respiration, blood pressure, heart rate and/or other such phenomena. Optionally and preferably, the system is small enough to be worn on the utility belt of an officer or security agent.

The present invention advances the field of PDD by delivering a digital polygraph, preferably portable, with an automated computer aided interrogation software system that will provide: 1) the time-controlled immersive virtual-reality (VR) presentation of multimedia stimuli composed of, for example, text, pictures, videos, sounds and/or sensations; and 2) the real-time analysis of the physical and psychophysiological responses of the examinee to these stimuli. The existence of a convenient, fast and preferably portable digital polygraph with such state-of-the-art psychometric analysis tools provides the opportunity to accelerate PDD use in passenger, witness, and testimony screening; in periodic espionage and sabotage testing; in law and judicial enforcement; and in other areas. Additionally, this intelligent deception verification system (IDVS) technology may assist interrogation researchers with state-of-the-art tools to improve deception detection methods and enhance their ability to detect the malicious intent of terrorists bent on harming others and/or property.

A preferred embodiment of such a system provides an improved human-computer interface (HCI) having many of the same capabilities as a conventional input device, like a keyboard, mouse or speech processor. A preferred embodiment may rely on or detect physiological signals from the brain and body, as well as from motion and vibration signals from the larynx, throat, tongue, or mouth.

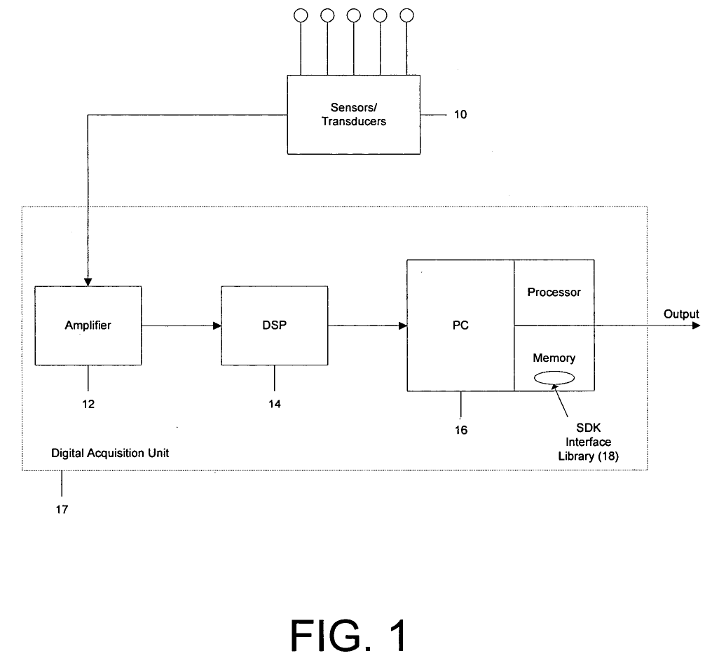

A preferred system embodiment of the HCI is illustrated in FIG. 1. As illustrated in FIG. 1, the system includes at least three primary parts: (1) a wearable sensor placement unit 10 (preferably stealthy and easy to don) that includes several transducer devices, such as the placement unit disclosed in FIGS. 1-6 and col. 4, line 54 to col. 6, line 60 of U.S. Pat. No. 5,038,782, to Gevins et al, which is incorporated herein by reference; (2) an integrated multichannel amplifier 12, a digital signal processing (DSP) unit 14 and a personal computer (PC) 16, preferably all small enough to wear on the human body; and (3) a virtual reality system 18. The PC 16 contains both a processing device and a memory. The amplifier 12 and/or DSP 14 may also be included within the housing of the PC 16 to miniaturize the overall system size, thereby producing an integrated digital acquisition unit 17. In a preferred embodiment, an Embla® recording device, produced by Flaga (Reykjavik, Iceland), may be used as the digital acquisition unit 17. Other data acquisition and processing devices, either alone or in combination, may be used and still be within the scope of the invention.

Preferably, the sensor placement unit 10 is capable of receiving electrophysiology in various forms, such as electroencephalographic (EEG) signals, electromyographic (EMG) signals, electrooculographic (EOG) signals, electrocardiographic (ECG) signals, as well as body position, motion and acceleration, vibration, skin conductance, respiration, temperature, and/or other physical measurements from transducers and/or other sensors. The system must be capable of delivering uncontaminated or substantially uncontaminated signals to the digital acquisition unit 17.

The sensor placement unit 10 preferably exhibits some or all of the following features: (1) it has relatively few input types (preferably less than eighteen, but it may include as many as forty or more) and can be quickly located on the body of the operator; (2) it positions biophysical (EEG, EOG, ECG, EMG, etc.) surface electrodes, and transducers for acquiring vibration, galvanic skin response (GSR), respiration, oximetry, motion, position, acceleration, load, and/or resistance, etc; (3) the sensor attachments are unobtrusive and easy to apply; (4) the sensor placement unit 10 accommodates multiple combinations of electrodes and/or transducers; (5) the surface electrodes use reusable and/or replaceable tacky-gel electrolyte plugs for ease of use and cleanliness; and optionally (6) EEG, EOG, ECG, and EMG electrodes may be positioned simultaneously and instantly on a human head and/or other body parts by a single positioning device.

In a preferred embodiment, the sensor placement unit 10 comprises a stealthy EEG placement system capable of also locating EOG, EMG, ECG, vibration, GSR, respiration, acceleration, motion and/or other sensors on the head and body. The sensor and transducer positioning straps preferably attach quickly and carry more than one type of sensor or transducer. In a preferred embodiment, the unit will include four EEG sensors, two EOG sensors, four EMG sensors, and a combination of vibration, acceleration, blood flow, GSR and position sensors. However, any combination of numbers and types of sensors and transducers may be used, depending on the application.

Each sensor may preferably be applied with the use of a semi-dry electrolyte plug with exceptional impedance lowering capabilities. In a preferred embodiment, a single electrolyte plug is placed onto each surface electrode and will enable the instantaneous collection of signals from the skin. Preferably, the electrolyte plugs are replaceable, and they may be used to rapidly record signal information from sensors without substantial, and preferably without any, abrasion or preparation of the skin. The electrolyte plugs should be removable to eliminate the need to immediately wash and disinfect the sensor placement unit 10 in liquids. By eliminating the need to wash the system after each use, the preferred sensor placement system 10 may be ideal for use in the home or office.

The sensor placement unit 10 preferably communicates with the digital acquisition unit 17, which includes an amplifier 12, a DSP 14 and a PC 16. The entire assembly preferably exhibits some or all of the following features: (1) it is small enough to wear on the body; (2) it has received Conformite Europeene (CE) marking and/or International Standards Organization (ISO) certification and is approved for use as a medical device in the United States; (3) it processes several, preferably at least sixteen and up to forty, multipurpose channels, plus dedicated event and video channels; (4) it provides a universal interface that accepts input from various sensors and powers several body-mounted transducers; (5) it is capable of high-speed digital signal processing of the EEG, EOG, ECG, EMG and/or other physiological signals; (6) it is capable of analyzing measurements from a host of transducer devices; and (7) it offers a suite of signal processing software for viewing and analyzing the incoming data in real time.

The digital acquisition unit 17 preferably provides an internal DSP system capable of performing real time cognitive, stress and motion assessment of continuous signals (such as EEG, EMG, vibration, acceleration, etc.) and generating spatial-frequency indexes, linear and non-linear data transforms and/or normalized data results. Processing requirements may include: (i) EOG detection and artifact correction; (ii) spatial, frequency and/or wavelet filtering; (iii) boundary element modeling (BEM) and finite element modeling (FEM) source localization; (iv) adaptive neural network pattern recognition and classification; (v) fast fuzzy cluster feature analysis methods; and (vi) real time generation of an output control signal derived from measures that may include (a) analysis of motion data such as vibration, acceleration, force, load, position, angle, incline and/or other such measures; (b) analysis of psychophysiological stress related data such as pupil motion, heart rate, blink rate, skin conductance, temperature, respiration, blood flow, pulse, and/or other such measures; (c) spatial, temporal, frequency and wavelet filtering of continuous physiological waveforms; (d) BEM and FEM based activity localization and reconstruction; (e) adaptive neural network pattern recognition and classification; and (f) fast fuzzy cluster feature extraction and analysis methods.

The data interface between the sensor placement system 10 and host PC 16 can be accomplished in a number of ways. These include a direct (medically isolated) connection or other connection such as via serial, parallel, SCSI, USB, Ethernet or Firewire ports. Alternatively, the data transmission from the sensor placement system 10 may be indirect, such as over a wireless Internet connection using an RF or IR link to a network card in the PCMCIA bay of the wearable computer.

The present invention preferably uses multimedia virtual-reality systems 18 and mathematically sophisticated cognitive and physiological signal processing and stress analysis utilizing highly constrained spatial-frequency pattern recognition techniques to provide innovative psychophysiological detection of deception methods.

A preferred embodiment of the present inventive IDVS interacts with the U.S. Army's wearable computing platform to provide broad interoperability with research-based and commercial interrogation systems and through compliance with the Advanced Distributed Learning Co-Lab's SCORM initiative. The U.S. Army specifies a Personal Armor System for Ground Troops (PASGT) with body armor, assault helmet and wearable computer that integrate weapon-mounted sensors and head-mounted displays. However, other platforms may also be used.

The present invention preferably provides: (1) a rapid use wearable digital polygraph with multimedia presentation capabilities; and (2) a programming environment that makes it easy for researchers and field examiners to create automated interrogation protocols that present multimedia stimuli (e.g., text, images, video clips, audio recordings, and tactile sensations) and automatically perform data analysis on a host of different signal types, which include but are not limited to, measures from the brain, heart, eyes, skin, muscles, voice, gestures and/or positions acquired by electrophysiological, electrodermal, thermal, vibratory, infra-red, laser, ultra-sound, video, motion and/or acceleration measurement devices.

By placing an individual into an immersive audio and visual virtual-reality environment 18 (within a large multimedia structure; by using portable VR glasses, such as those used in virtual reality games; by using an auditory system, such as headphones, and or by using a haptic system used to convey information to the examinee through the skin, such as from a small vibrating pen or movement of a chair), the novel environment may, minimally, place cognitive demands on the examinee that disrupt his or her attempts to conceal the use of mental and physical countermeasures used to defeat detection of deception. Immersive multimedia virtual reality (IMVR) 18 may lead to vastly improved methods of deception detection and may play a significant role in computer-aided interrogation and psychophysiological detection of deception technologies.

For example, an IMVR system 18 may present stimuli that the examinee perceives as placing the examinee on a moving rollercoaster. By providing, at least one of visual, audio and tactile stimuli to the examinee, the IMVR environment 18 may distract the examinee and limit the examinee's ability to use countermeasures to defeat detection of deception.

Furthermore, the INMVR system 18 may present stimuli depicting, for example, one or more images of a crime scene, a weapon used in a crime, an individual involved in a crime (i.e., another participant in the commission of the crime or a victim) or other images. An examinee's psychophysiological reaction to the image may be monitored to determine whether the examinee has previously seen the image. For example, if an image of a murder scene is presented to an examinee that did not commit the murder, the examinee may be expected to exhibit an expected reaction, such as shock, upon viewing the scene. However, an examinee that had previously witnessed the murder scene (presumably because the examinee had been a participant in the crime) may exhibit no reaction or a less pronounced or different reaction than what might otherwise be expected. The digital acquisition unit 17 may record psychophysiological input signals during the presentation of the image and report to the examiner whether the examinee exhibited the expected reaction when the image was presented. Alternatively, the IMVR system 18 may present stimuli affecting other senses, such as sounds, smells, flavors, and/or tactile sensations, in order to evoke reactions from the examinee.

To deal with the problem of physical countermeasures, a preferred embodiment may include methods and systems for monitoring brainwaves; eye, heart and/or muscle activity; temperature; skin conductance, resistance, and/or impedance; body position, posture, expression, and/or gestures; motion; speech; blood flow and volume; and/or stress indicating measures like respiration, blood pressure, heart rate, and/or other such phenomena that can be sensed from the body, either in contact or from a distance. In particular, muscle activity from the ankles (to detect toe curls) and from the throat, tongue or larynx (to detect tongue biting, as well as to record voice stress patterns) may be useful. IMVR techniques may be used to combat physical and more complicated mental countermeasures such as counting, imagined pattern manipulation or other such cognitive processing schemes. Hence, a preferred embodiment may integrate a wide variety of sensor technologies within a digital polygraph framework that includes computer aided stimulus presentation and automated multimodal signal analysis capabilities.

A preferred embodiment may apply immersive three-dimensional multimedia virtual-reality stimulus delivery techniques 18, expert-based digital signal processing algorithms, and adaptive neural network (ANN) digital signal classification and recognition techniques to process multimodal psychometric signals and improve the accuracy of the present invention over traditional PDD methods. Preferably, the signal processing algorithms may examine the power of the signals received from the wearable sensor unit 10 in the frequency domain. Frequencies of interest may be chosen based on the deception technique to be detected and the placement of the sensor. Preferably, the frequency domain of interest is between 1 and 40 Hz. The PC 16 or an electrically or wirelessly connected processing unit may perform spatial-frequency analysis by analyzing the selected frequencies and the interaction among signals from different sensors. Spatial-frequency analysis may be used to determine measures of, for example, executive load, arousal, engagement, attention and stress.

Additionally, the present invention may substantially or completely remove the ambiguity of examiner subjectivity by automating the presentation of questions, as well as the analysis normally carried out by the examiner. Further, by virtually manipulating the visual, auditory and/or haptic environment of the examinee, the present invention may prevent the successful use of countermeasures to defeat detection. The technology embodied in the present invention may be accomplished by coupling cognitive neuroscience and mathematical signal processing methods with immersive 3D graphical visualization tools and robust audio synthesizers 18 to create an inimitable multimodal environment that distracts and redirects the mental and stress related processes of the examinee, thereby disrupting the internal cognitive framework of the examinee.

To provide true portability and reliability, a preferred embodiment of the present invention may provide wireless Web-enabled data transmission capabilities to upload examinee data onto a secure website for real-time examination by domain-specific experts, if needed. In a preferred embodiment, the entire system may be small enough to be carried on a utility belt and may provide easy to use multi-sensor assemblies to locate the sensors, transducers, cameras, and other such imaging devices on, near, or in the proximity of the examinee.

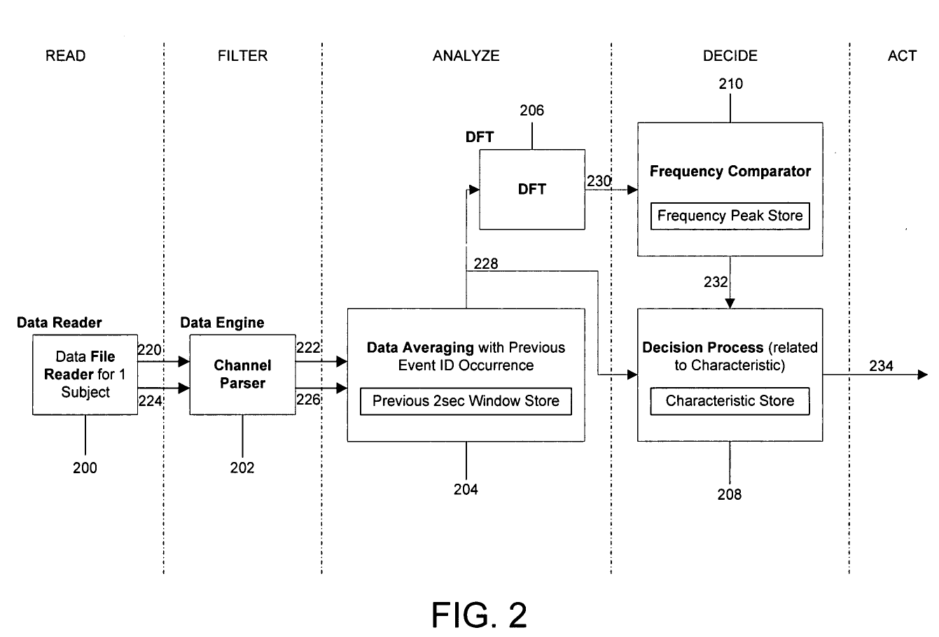

FIG. 2 illustrates an exemplary agent flow control diagram according to an embodiment of the present invention. The objective of the exemplary algorithm is to locate consistent frequency peaks in the information supplied by the sensor placement unit 10 and to determine whether such peaks indicate deception by a test subject. Initially, the data acquisition unit 17 reads initial channel data from the sensor placement unit 10 using a data reader 200. The initial channel data may be used to initialize the data acquisition unit 17. The data 220 may be transmitted to a data engine 202 that filters the information on a per channel basis. The data 220 may include a time stamp and a list of the channel names and types from the sensor placement unit 10. The data engine 202, for each channel, may then send the filtered channel information 222 to a data averaging unit 204. The filtered channel information 222 may include a time stamp, the channel name and the initial data for the channel.

After initialization is complete, data may be received from the sensor placement unit 10 as required. The data reader 200 may load the received data and forward 224 it to the data engine 202. The forwarded data 224 may include a time stamp and a list of data for all channels. The data engine 202 may filter the information by channel and, for each channel, send filtered data 226 to a data averaging unit 204. The data averaging unit 204 may maintain a buffer of filtered data on a per channel basis for a given time period, such as the previous two seconds. The data averaging unit 204 may perform cumulative data averaging on the buffered data and send the resulting information (buffered data) 228 to the DFT 206 and the decision process module 208. The buffered data 228 may include a time stamp, the number of points of information, and the cumulative average of the information. The DFT 206 may create frequency data for the signals from the buffered data 228 by analyzing the frequency between peaks of the buffered data. The DFT 206 may send frequency data 230, such as a time stamp and frequency peak information, to a frequency comparator 210. The frequency comparator 210 may store the frequency peak information in a frequency peak buffer. The decision process module 208 may use the frequency peak buffer values 232 and the buffered data 228 to determine a characteristic 234. The characteristic 234 may determine whether the data acquisition unit 17 believes that the test subject is attempting to deceive the IDVS.

For example, a series of readings may be taken for an examinee over a period of time, such as two minutes, in order to generate a baseline or average value for each input signal. The readings may be based on questions presented to the examinee in a “normal” environment (i.e., an environment in which the IMVR system 18 is not presenting stimuli designed to evoke a reaction, distract the examinee, or otherwise prevent the examinee from evading detection of deception). The examinee may then be presented with IMVR stimuli simulating a novel environment designed to detect deception by distracting or evoking a reaction from the examinee. The examinee may be questioned while the IMVR environment is active. The values for the input signals during the period when the non-normal environment is presented may be compared to the baseline values for each signal in order to determine whether the examinee is attempting to evade detection of deception.

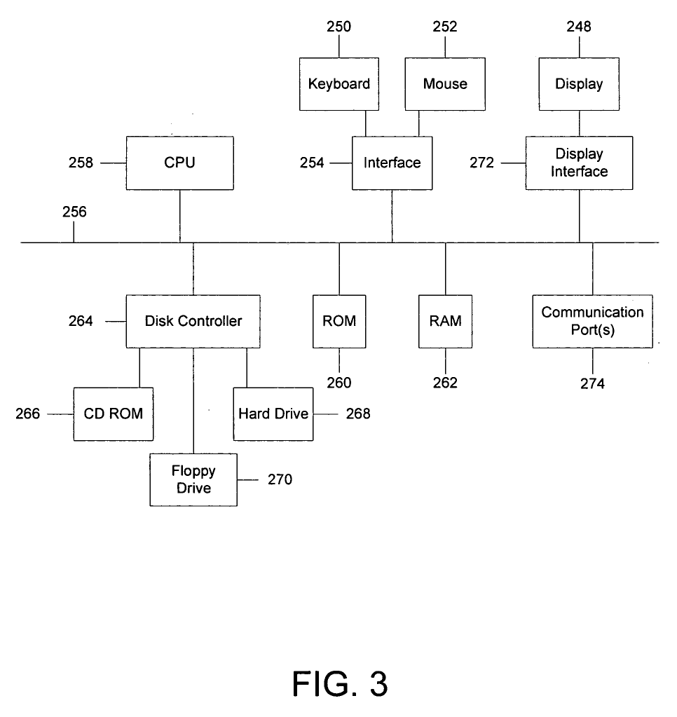

FIG. 3 is a block diagram of exemplary internal hardware that may be used to contain or implement the program instructions of a system embodiment of the present invention. Referring to FIG. 3, a bus 256 serves as the main information highway interconnecting the other illustrated components of the hardware. CPU 258 is the central processing unit of the system, performing calculations and logic operations required to execute a program. Read only memory (ROM) 260 and random access memory (RAM) 262 constitute memory devices.

A disk controller 264 interfaces one or more optional disk drives to the system bus 256. These disk drives may be external or internal floppy disk drives such as 270, external or internal CD-ROM, CD-R, CD-RW or DVD drives such as 266, or external or internal hard drives 268. As indicated previously, these various disk drives and disk controllers are optional devices.

Program instructions may be stored in the ROM 260 and/or the RAM 262. Optionally, program instructions may be stored on a computer readable carrier such as a floppy disk or a digital disk or other recording medium, a communications signal, or a carrier wave.

An optional display interface 272 may permit information from the bus 256 to be displayed on the display 248 in audio, graphic or alphanumeric format. Communication with external devices may optionally occur using various communication ports such as 274.

In addition to the standard computer-type components, the hardware may also include an interface 254 which allows for receipt of data from the sensors or transducers, and/or other data input devices such as a keyboard 250 or other input device 252 such as a remote control, pointer, mouse, joystick, and/or sensor/transducer input.

The many features and advantages of the invention are apparent from this description. However, since numerous modifications and variations will readily occur to those skilled in the art, it is not desired to limit the invention to the exact construction and operation illustrated and described. Accordingly, all suitable modifications and equivalents may be included within the scope of the invention.

What is claimed is:

1. A deception verification system, comprising:
a sensor placement unit having a plurality of sensors;
a digital acquisition unit that receives signals from the sensor placement unit, wherein the digital acquisition unit includes:
one or more multichannel amplifiers,
one or more digital signal processing units, and
a computing unit having one or more processing devices and one or more memories; and
a virtual reality system.

2. The deception verification system of claim 1 wherein the sensor placement unit is wearable.

3. The deception verification system of claim 1 wherein the sensor placement unit has approximately eighteen to approximately forty-two sensors, wherein one sensor includes an event channel, wherein one sensor includes a video channel, wherein each remaining sensor includes a multipurpose channel.

4. The deception verification system of claim 1 wherein the plurality of sensors receive physiological signals including one or more of electroencephalographic (EEG) signals, electromyographic (EMG) signals, electrooculographic (EOG) signals, electrocardiographic (ECG) signals, body position, motion and acceleration, vibration, skin conductance, respiration, and temperature.

5. The deception verification system of claim 1 wherein each of the plurality of sensors includes a surface electrode with an electrolyte plug.

6. The deception verification system of claim 5 wherein each electrolyte plug is removably attached to the surface electrode.

7. The deception verification system of claim 1 wherein the digital acquisition unit is wearable.

8. The deception verification system of claim 1 wherein the digital acquisition unit performs real-time cognitive, stress and motion assessments of continuous signals received from the plurality of sensors and generates one or more of spatial-frequency indices, linear or non-linear data transforms, and normalized data results.

9. The deception verification system of claim 1 wherein the virtual reality system includes at least one of virtual reality glasses, an auditory system and a haptic system.

10. The deception verification system of claim 1 wherein the virtual reality system includes a structure containing auditory and visual systems.

11. A deception verification system, comprising:
one or more sensor placement units, wherein each sensor placement unit comprises a plurality of transducer devices;
one or more multichannel amplifiers, wherein each amplifier receives one or more signals from at least one sensor placement unit;
one or more digital signal processing units, wherein each digital signal processing unit receives amplified signals from at least one multichannel amplifier;
a first computing unit having one or more processing devices and one or more memories, wherein the computing unit receives processed signals from at least one digital signal processing unit; and
a virtual reality system.

12. The deception verification system of claim 11, further comprising a second computing unit having one or more processing devices and one or more memories.

13. The deception verification system of claim 12 wherein the one or more memories of the first computing unit contain instructions for performing the following:
sending commands to the virtual reality system to generate one or more stimuli;
receiving one or more signals from the one or more digital signal processing units representative of physiological occurrences; and
sending data to the second computing unit representative of the one or more signals.

14. The deception verification system of claim 13 wherein the one or more memories of the second computing unit contain instructions for performing the following:
receiving the data from the first computing unit;
performing spatial-frequency analysis on the data to obtain information regarding the likelihood of deception; and
sending the information to the first computing unit.

15. The deception verification system of claim 12 wherein the second computing unit is wirelessly connected to the first computing unit.

16. The deception verification system of claim 12 wherein the second computing unit is electrically connected to the first computing unit.

17. A method of performing deception verification, comprising:
stimulating one or more senses of an examinee with a virtual reality system;
questioning the examinee;
determining psychophysiological data from the examinee using a plurality of sensors;
analyzing the psychophysiological data; and
determining a likelihood of deception by the examinee.

18. The method of claim 17 wherein at least one of the plurality of sensors is placed on the skin of the examinee.

19. The method of claim 17 wherein analyzing the psychophysiological data is performed using one or more computers.

20. The method of claim 19 wherein at least one of the computers includes a program containing instructions for performing one or more of the following:
electrooculographic detection;
artifact correction;
spatial filtering;
frequency filtering;
wavelet filtering;
boundary element modeling source localization;
finite element modeling source localization;
adaptive neural network pattern recognition; and
fast fuzzy cluster feature analysis.

21. The method of claim 17 wherein analyzing the psychophysiological data comprises:
receiving one or more signals at one or more frequencies for each sensor;
determining a power amplitude of each signal for each sensor; and
analyzing one or more relationships between the power amplitudes for one or more signals from one or more sensors at one or more frequencies.

22. The method of claim 21 wherein each of the one or more frequencies is between approximately 1 Hz and approximately 40 Hz.

23. The method of claim 17 wherein analyzing the psychophysiological data comprises determining values for one or more of the following:
high-order executive workload;
arousal;
engagement;
attention; and
stress.

24. The method of claim 17 wherein the virtual reality system includes at least one of the following:
virtual reality glasses for directing visual stimuli to the examinee;
an auditory system for directing audio stimuli to the examinee; and
a haptic system for directing tactile stimuli to the examinee.

25. The method of claim 17 wherein the virtual reality system includes a structure containing at least one of:
an auditory system for directing audio stimuli to the examinee;
a visual system for directing visual stimuli to the examinee; and
a haptic system for directing tactile stimuli to the examinee.

26. The method of claim 17 wherein determining the likelihood of deception is based at least in part on presenting one or more particular stimuli using the virtual reality system.

27. A deception verification system, comprising:
a sensor placement unit having a plurality of sensors;
a digital acquisition unit that receives signals from the sensor placement unit; and
a virtual reality system.

28. The deception verification system of claim 27 wherein the virtual reality system presents one or more stimuli tailored to an examinee.
